(19) United States
(12) Patent Application Publication
(10) Pub. No.: US 2012/0215160 A1
(43) Pub. Date: Aug. 23, 2012

(54) OPHTHALMIC SURGICAL SYSTEMS HAVING INTRAOCULAR PRESSURE STABILIZING APPARATUS

(76) Inventors: Michael J. Valenti, Rochester, NY (US); Brian D. McCary, Clayton, MO (US); Joseph S. Rosenshein, Rochester, NY (US)

(21) Appl. No.: 13/331,194

(22) Filed: Dec. 20, 2011

Related U.S. Application Data
(60) Provisional application No. 61/428,887, filed on Dec. 31, 2010.

(51) Int. Cl. A61F 9/007; A61M 1/00

(52) U.S. Cl. 604/31; 604/66

(57) ABSTRACT

An ophthalmic surgical apparatus for use with an eye including a tube adapted to be in fluid communication with the eye, a pressure sensor adapted to measure an intraocular pressure value of the eye, a fluid reservoir comprising a moveable wall, the fluid reservoir adapted to be in fluid communication with the eye through the tube, and at least one processor coupled to the pressure sensor to receive the measured intraocular pressure value, the at least one processor operable to position the moveable wall in response to a difference between the measured intraocular pressure value and a target intraocular pressure value.

### CROSS REFERENCE

This application claims the benefit of Provisional Patent Application No. 61/428,887 filed Dec. 21, 2010 which is incorporated by reference herein.

### FIELD OF INVENTION

The present invention relates to ophthalmic surgical systems, and more particularly to ophthalmic surgical systems having intraocular pressure (IOP) stabilizing apparatus.

### BACKGROUND OF THE INVENTION

The lens of a human eye may develop a cataracteous condition which affects a patient's vision. Cataracteous lenses may be fragmented and removed using a surgical apparatus in a procedure commonly referred to as a lensectomy. Lens fragmentation can be achieved using ultrasound in a phacoemulsification lensectomy (also referred to simply as “phaco”), laser lensectomy or other procedures. Removal of a fragmented lens is typically performed using one or more hand pieces which perform irrigation and/or aspiration. In FIG. 1, a hand piece 12 is shown that has a tip 14 that is inserted through an incision in the cornea 16 for performing irrigation and aspiration. Such a hand piece is typically connected to a surgical console 20 which allows a surgical staff member to control irrigation and aspiration as well as various other parameters of the surgical system 10 such as those related to ultrasound or laser performance.

The broken lens is removed through an aspiration line 40 that is coupled between the hand piece and a vacuum source 46. The distal end of the tip has an opening that is in fluid communication with the aspiration line. The distal end of the tip also typically has a sleeve which has an opening in fluid communication with an irrigation line 28. The irrigation line is typically connected to an irrigation source 30 that can provide irrigation fluid to the surgical site.

The lens pieces and irrigation fluid are drawn into the aspiration line through the opening of the tip. When performing an irrigation and aspiration procedure, it is typically desirable to maintain a suitable, positive intraocular pressure (IOP) within the eye. Insufficient or elevated intraoperative IOP may increase the incidence of complications during surgery and may also affect the incidence of postoperative complications.

The fluctuations in pressure during surgery have many causes. Surgical tool manipulation can cause large pressure increases with long durations. Occlusion of aspiration tools and post occlusion surges can cause significant pressure spikes that have rise times in the millisecond range. Clearance of these same occlusions can cause pressure dips that may lead to momentary chamber collapse and rupture of the posterior chamber capsule, resulting in a need for additional surgery.

### SUMMARY

Aspects of the present invention are directed to an ophthalmic surgical apparatus for use with an eye, comprising a tube adapted to be in fluid communication with the eye, a pressure sensor adapted to measure an intraocular pressure value of the eye, a fluid reservoir comprising a moveable wall, the fluid reservoir adapted to be in fluid communication with the eye through the tube, and at least one processor coupled to the pressure sensor to receive the measured intraocular pressure value, the at least one processor operable to position the moveable wall in response to a difference between the measured intraocular pressure value and a target intraocular pressure value.

In some embodiments, the apparatus further comprises a fluid source adapted to provide fluid to the eye through the tube. In some embodiments, the apparatus further comprises a vacuum source adapted to draw fluid from the eye through the tube.

The pressure sensor may be disposed within the tube. The pressure sensor may be a dual sensor, non-invasive pressure sensor.

In some embodiments, the moveable wall comprises a flexible membrane. In some embodiments, the moveable wall constitutes a wall of an accordion-shaped container.

The apparatus may comprise a pump fluidly coupled between the fluid source and the reservoir.

The apparatus may further comprise a voice coil, wherein the at least one processor is operable to position the moveable wall using the voice coil. In some embodiments, the apparatus further comprises a stepper motor, wherein the at least one processor is operable to position the moveable wall using the stepper motor.

The apparatus may further comprise a second tube adapted to be in fluid communication with the eye, a vacuum source adapted to draw fluid from the eye through the second tube, a second fluid reservoir comprising a second moveable wall, the second fluid reservoir adapted to be in fluid communication with the eye through the second tube, and the at least one processor coupled to the pressure sensor to receive the measured intraocular pressure value, the processor operable to position the second moveable wall in response to the difference between the measured intraocular pressure value and the target intraocular pressure value.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1 is a partial schematic illustration of a conventional, surgical apparatus including an irrigation line and an aspiration line;

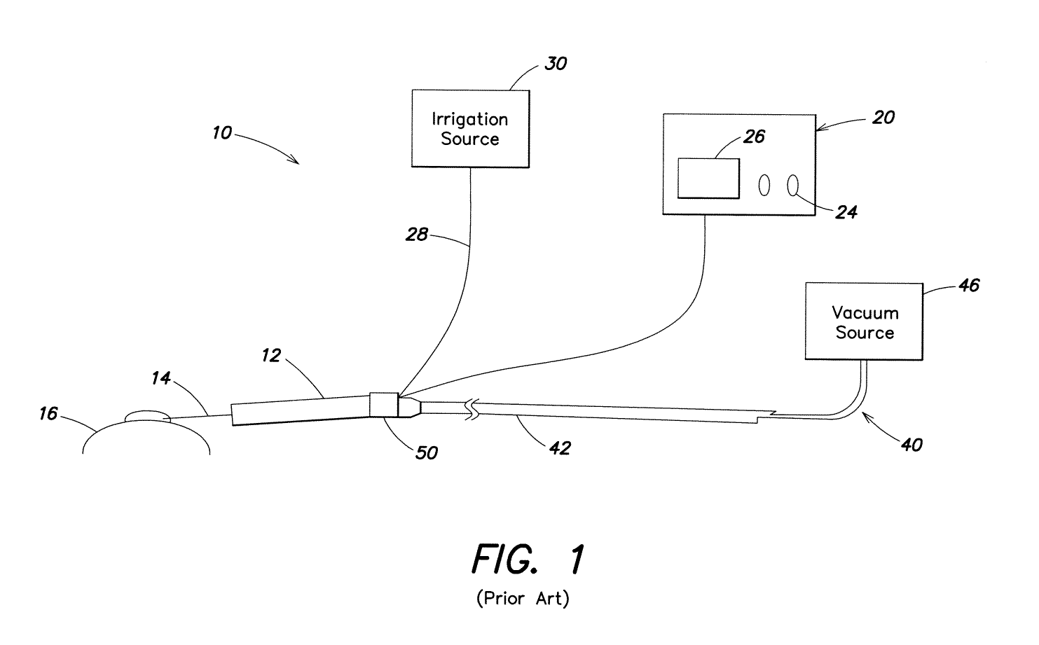

FIG. 2 is partial schematic illustration of an example of a surgical apparatus according to aspects of the present invention comprising an irrigation line providing pressure stabilization;

FIGS. 3A-3C are schematic illustrations of examples of actuation devices suitable for use in providing pressure stabilization according to aspects of the present invention; and

FIG. 4 is a partial schematic illustration of another example of a surgical apparatus according to aspects of the present invention comprising an irrigation line providing pressure stabiliatoin and an aspiration line providing pressure stabilization.

### DETAILED DESCRIPTION

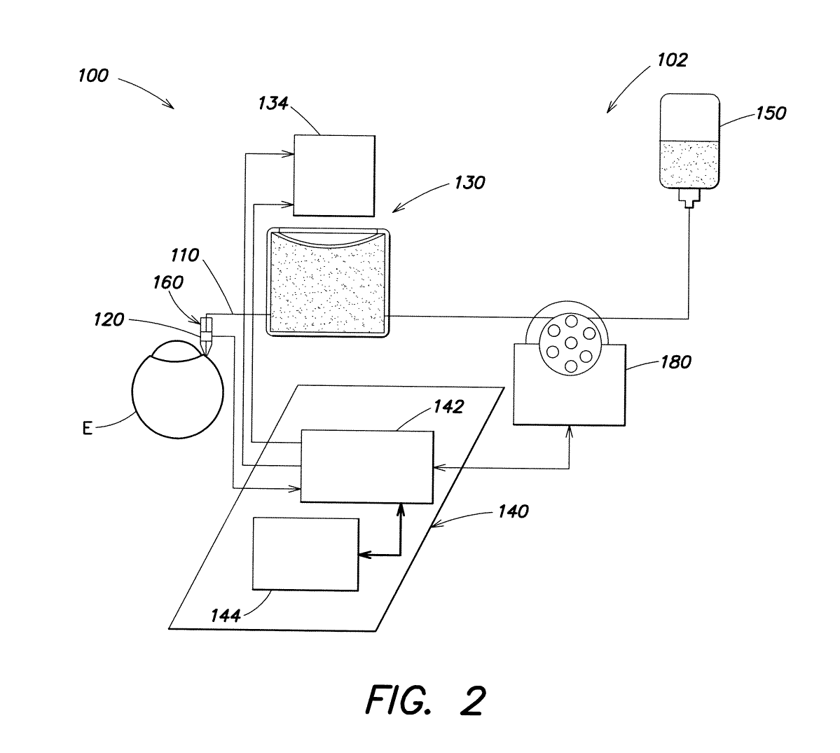

FIG. 2 is a partial schematic illustration of an example of a surgical apparatus 100 according to aspects of the present invention comprising an irrigation system 102 providing pressure stabilization. System 102 comprises irrigation tube 110, a pressure sensor 120, a fluid reservoir 130, and a processor 140 for processing IOP information.

Irrigation tube 110 is adapted to be in fluid communication with the eye E. The tube is connected between a fluid source 150 (e.g., a bottle or other container of buffered saline solution) and eye E. The tube is sized and shaped to provide suitable fluid flow and fluid pressure in the eye. In the illustrated embodiment, the irrigation tube is adapted to be in fluid communication with the eye through a hand piece 160.

Pressure sensor 120 is adapted to measure an intraocular pressure value the eye E. Any suitable pressure sensor capable of providing the processor with an IOP value may be used. In some embodiments, the pressure sensor is disposed in the fluid path of the irrigation system. It will be appreciated that it is generally advantageous that a sensor in the fluid path be located proximate the eye, so that the measured value accurately represents the IOP. For example, the sensor may be disposed in hand piece 160. A sensor to be placed in the fluid path may, for example, be a non-invasive, dual transducer device as described in U.S. Pat. No. 5,865,764, to Moorehead, titled DEVICE AND METHOD FOR NONINVASIVE MEASUREMENT OF INTERNAL PRESSURE WITHIN BODY CAVITIES, issued Feb. 2, 199, the substance of which is hereby incorporated by reference.

Fluid reservoir 130 is in fluid communication with the irrigation tube and comprises a moveable wall 132. The reservoir contains a biocompatible liquid such as buffered saline solution that is present in the fluid source. The reservoir contributes to a baseline fluid pressure in eye E when the diaphragm is stationary for a sufficient time to attain an ambient pressure. However, according to aspects of the present invention, actuator 134 moves the movable wall to modify the pressure in the eye in response to IOP values measured by sensor 120 during eye surgery. It will be appreciated that, in addition to the moveable wall, the remainder of the reservoir is sufficiently rigid such that a pressure change in the eye can be attained in response to movement of the moveable wall. For example, the moveable wall may comprise a flexible diaphragm in an otherwise rigid container or may comprise an accordion-shaped container where opposing walls are moved relative to one another.

Processor 140 is coupled to pressure sensor 120 to receive the measured intraocular pressure value. The processor is operable to position moveable wall 132 in response to a difference between the measured intraocular pressure value and a target intraocular pressure value. Although in the illustrated embodiment the processor is shown as comprising a system processor 144 (e.g., a processor in a conventional surgical console (e.g., for receiving using inputs such as pump speed), such as the processor in the Stellaris®, available from Bausch and Lomb Incorporated, Rochester, N.Y.) and a chamber stability processor 142 (e.g, a processor capable of providing signals to and from sensor, processor, and actuator in the manner set forth herein), any suitable processor or processors may be used to receive and send signals to each of relevant components.

In some embodiments, a variable-speed infusion pump 180 is fluidly coupled between fluid source and reservoir. The pump operates to inject fluid into the reservoir between actuation events to return the diaphragm to a nominal position thereby increasing response rate of the system and permitting greater precision in the response that occurs when pressure is adjusted.

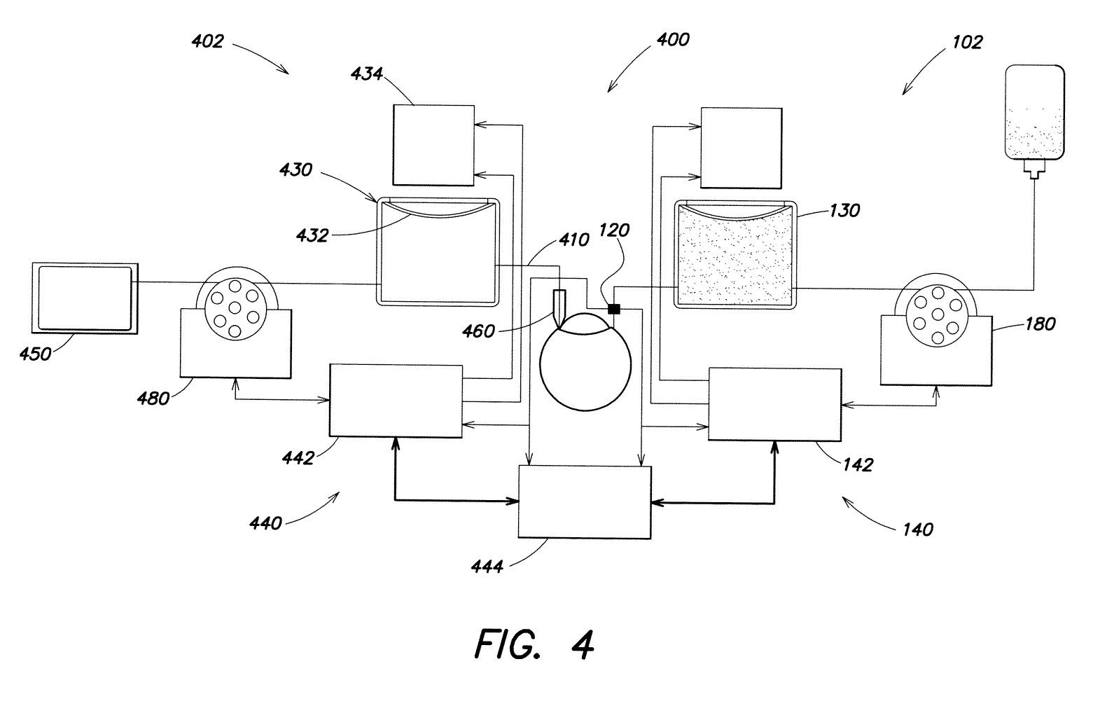

Although the illustrated embodiment of a pressure stabilizer is shown in conjunction with an irrigation system, it will be appreciated that the pressure stabilizer can be implemented in an aspiration system, for example, as shown in FIG. 4 below. It will also be appreciated that, although the irrigation tube is adapted to be in fluid communication with the eye through a hand piece, the tube can be connected to another instrument (not shown) which in turn is in fluid communication with an eye or the tube can be configured to be inserted directly into an eye. It will also be appreciated that a pressure stabilizer as described herein can be used in apparatus to perform anterior segment surgery (e.g., cataract surgery) or posterior segment surgery (e.g., vitrealretinal surgery).

In use during surgery, apparatus 100 provides irrigation to an eye from fluid source 150 in a conventional manner while measuring IOP using sensor 120. Upon measurement of a pressure that is outside of a range, processor 140 causes actuation device 134 to move a wall of reservoir 130. In response to a measured pressure that is too low, the wall is moved inward. Because the fluid in the reservoir is incompressible the fluid flows into tube 110 and then into the eye, thereby providing a compensatory increase in IOP. It will be appreciated that pump 180 can operate to prevent all flow between the reservoir and the fluid source; and in embodiments where the pump is omitted a valve (not shown) (e.g., under control of processor 140) can be provided between reservoir 130 and fluid source 150 to control flow between the reservoir and the source. In response to a measured pressure that is too high, the wall is moved outward drawing fluid form the eye, thereby providing a compensatory decrease in IOP. It will be appreciated that pump 180 or the valve can be operated to prevent flow from coming from the fluid source.

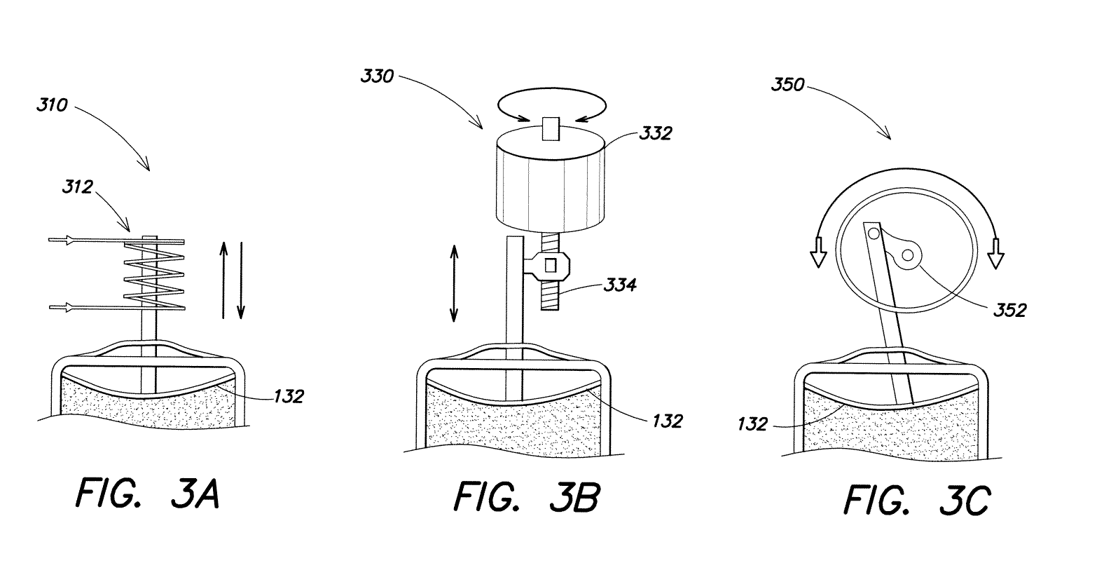

FIGS. 3A-3C are schematic illustrations of examples of actuation devices 310, 330, 350 suitable for use in pressure stabilizers according to aspects of the present invention. Each actuation device comprises a movement mechanism for moving movable wall 132. In FIG. 3A, the actuation device is embodied as a voice coil 312. In FIG. 3B, the actuation device is embodied as a stepper motor 332 on a lead screw 334. In FIG. 3C, the actuation device is embodied as a stepper motor 352 on a cam.

FIG. 4 is partial schematic illustration of an example of a surgical apparatus according to aspects of the present invention comprising an irrigation system providing pressure stabilization and an aspiration system providing pressure stabilization.

Surgical apparatus 400 comprises an irrigation system 102 as described above with reference to FIG. 2 and an aspiration system 402 providing pressure stabilization. System 402 comprises aspiration tube 410, a pressure sensor 420, a fluid reservoir 430, a vacuum source 450, and a processor 440 (comprising vacuum processor 442 and, in part, system processor 444) for processing IOP information. In the illustrated embodiment, processor 444 receives and/or processes aspiration information and irrigation information (e.g., user inputs related to the speed of pumps 480 and 180); however separate aspiration and irrigation system processors could be used. To process irrigation information, processor 444 operates with processor 442 in the manner of processor 144 as described above.

Aspiration tube 410 is adapted to be in fluid communication with the eye E. The tube is connected between vacuum source 450 and eye E. The tube is sized and shaped to provide a suitable fluid flow and fluid pressure in the eye. In the illustrated embodiment, the aspiration line is adapted to be in fluid communication with the eye through a hand piece 460. Although the aspiration and irrigation tubes are shown as extending through separate hand pieces, in some embodiments both tubes extend through a common handpiece.

Pressure sensor 120 is adapted to measure an intraocular pressure value of eye E as described above. Although the pressure sensor is shown as positioned to determine IOP using irrigation fluid in an irrigation tube, in other embodiments, a pressure sensor can be positioned to determine IOP using aspiration fluid in an aspiration tube. It will be appreciated that using irrigation flow may be advantageous since the aspiration tube may be come occluded during removal of a cataract.

Fluid reservoir 430 is in fluid communication with the aspiration tube and comprises a moveable wall 432. The reservoir contributes to a baseline fluid pressure in eye E when the diaphragm is stationary for a sufficient time to attain an ambient pressure. However, according to aspects of the present invention, actuator 434 moves the movable wall to modify the pressure in the eye in response to IOP values measured by sensor 420 during eye surgery. Similar to fluid reservoir 130 described above, in addition to the moveable wall, the remainder of reservoir 430 is sufficiently rigid such that a pressure change in the eye can be attained in response to movement of the moveable wall. The moveable wall 432 may comprise a flexible diaphragm.

Processor 440 is coupled to pressure sensor 420 to receive the measured intraocular pressure value. The processor is operable to position moveable wall 432 in response to a difference between the measured intraocular pressure value and a target intraocular pressure value. Although in the illustrated embodiment the processor is shown as comprising a system processor 444 (e.g., a processor in a conventional surgical console, such as the processor in the Stellaris, available from Bausch and Lomb Incorporated, Rochester, N.Y.) and a chamber stability processor 442 (e.g., a processor capable of providing signals to and from sensor 120 and actuator 434 in the manner set forth herein), any suitable processor or processors may be used to receive and send signals to each of the relevant components.

Vacuum pump 480 is fluidly coupled between vacuum cassette 450 and reservoir 430. The pump operates to draw fluid from the eye to cassette 450 in a conventional manner.

In use during surgery, apparatus 400 provides irrigation to an eye the manner described above with reference to FIG. 2 while measuring IOP using sensor 120 and responding when necessary by moving a wall of reservoir 130.

Also during surgery, apparatus 400 aspirates the eye in a conventional manner while IOP is measured using sensor 120. Upon measurement of a pressure that is outside of a range, processor 440 causes actuation device 434 to move a wall of reservoir 430. In response to a measured pressure that is too low, the wall is moved inward. Because the fluid in the reservoir is incompressible, the fluid flows into tube 410 and toward the eye, thereby providing a compensatory increase in IOP. In addition, following release of an occlusion, fluid is provided into the aspiration line toward the pump thereby preventing severe negative pressure from building up in the aspiration line, thereby helping to alleviate what is commonly referred to as post occlusion surge. In response to a measured pressure that is too high, the wall is moved outward drawing fluid form the eye, thereby providing a compensatory decrease in IOP.

It will be appreciated that, although the aspiration line is adapted to be in fluid communication with the eye through a hand piece, the tube can be connected to another instrument (not shown) which in turn is in fluid communication with an eye or the tube can be configured to be inserted directly into an eye.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An ophthalmic surgical apparatus for use with an eye, comprising:
a tube adapted to be in fluid communication with the eye;
a pressure sensor adapted to measure an intraocular pressure value of the eye;
a fluid reservoir comprising a moveable wall, the fluid reservoir adapted to be in fluid communication with the eye through the tube; and
at least one processor coupled to the pressure sensor to receive the measured intraocular pressure value, the at least one processor operable to position the moveable wall in response to a difference between the measured intraocular pressure value and a target intraocular pressure value.

2. The apparatus of claim 1, further comprising a fluid source adapted to provide fluid to the eye through the tube.

3. The apparatus of claim 1, further comprising a vacuum source adapted to draw fluid from the eye through the tube.

4. The apparatus of claim 1, wherein the pressure sensor is disposed within the tube.

5. The apparatus of claim 4, wherein the pressure sensor is a dual sensor, non-invasive pressure sensor.

6. The apparatus of claim 1, wherein the moveable wall comprises a flexible membrane.

7. The apparatus of claim 1, wherein the moveable wall constitutes a wall of an accordion-shaped container.

8. The apparatus of claim 2, further comprising a pump fluidly coupled between the fluid source and the reservoir.

9. The apparatus of claim 1, further comprises a voice coil, wherein the at least one processor is operable to position the moveable wall using the voice coil.

10. The apparatus of claim 1, further comprises a stepper motor, wherein the at least one processor is operable to position the moveable wall using the stepper motor.

11. The apparatus of claim 2, further comprising:
a second tube adapted to be in fluid communication with the eye;
a vacuum source adapted to draw fluid from the eye through the second tube;
a second fluid reservoir comprising a second moveable wall, the second fluid reservoir adapted to be in fluid communication with the eye through the second tube; and
the at least one processor coupled to the pressure sensor to receive the measured intraocular pressure value, the processor operable to position the second moveable wall in response to the difference between the measured intraocular pressure value and the target intraocular pressure value.
